(19) United States
(12) Patent Application Publication
(10) Pub. No.: US 2004/0072915 A1
(43) Pub. Date: Apr. 15, 2004

(54) Novel medicines based on sesquiterpene mixtures

(76) Inventors: Andre Rougereau, Tours (FR); Odile Rougereau-Person, Tours (FR)

Correspondence Address: CONLEY ROSE, P.C., P. O. BOX 3267, HOUSTON, TX, 77253-3267, US

(21) Appl. No.: 10/258,667

(22) PCT Filed: Apr. 27, 2001
(86) PCT No.: PCT/FR01/01327

(30) Foreign Application Priority Data
Apr. 27, 2000 (FR) 00/05403

(51) Int. Cl. A61K 31/045

(52) U.S. Cl. 514/729

(57) ABSTRACT

The invention concerns pharmaceutical compositions characterized in that they contain, in association with a pharmaceutically inert carrier, a therapeutically effective amount of a mixture of α-bisabolol and farnesol and/or geraniol.

Application in particular in the prevention and treatment of cancers.

The invention relates to new drugs based on mixtures of sesquiterpenes and/or derivatives of sesquiterpenes.

Sesquiterpenes are formed from repetitions of C₅ isoprenoid units, many of them constituting the active ingredients of natural essential oils.

Prior work by inventors had shown the existence of derivatives of such compounds, in particular geranyl/farnesyl, in extracts obtained from tumour masses (the primary tumour actual tumoural source or metastasic source).

These results were compared to experimental data showing the value of C₁₅ and C₂₀ isoprenoids in the formation of proteic derivatives related to the tumour cells, such as protein ras 21. Blocking the farnesylation of proteins could thus enable to transform oncogenic activity of protein ras 21 and subsequently destroy the tumour cell by apoptosis.

The inventors have now observed the synergy of certain sesquiterpene mixtures of great interest in oncology or derivatives of sesquiterpenes, for specific therapeutic indications. These therapeutic indications are related to the specific accumulation of these sesquiterpenes in tissue masses undergoing rapid renewal either physiological or pathological, such as tumour masses.

The aim of the invention is thus to provide never drugs with a specific anti-tumour effect, containing mixtures of sesquiterpenes and/or derivatives of sesquiterpenes. The term sesquiterpene, such as used in the description and the claims, will equally designate the sesquiterpenes themselves or their derivatives.

According to another aspect, it also aims at the use of these compositions to prepare drugs for the prevention and/or treatment of epithelial or connective tissue.

According to the invention, the compositions are characterized in that they contain, optionally in association with an inert pharmaceutical vehicle, a therapeutically effective amount of a mixture of α-bisabolol and farnesol and/or geraniol, and/or their derivatives, particularly functional derivatives such as their esters, for example, the methohexate, their ethers, in particular with hydroxylated compounds such as adriamycin, C1-C4 alkyl ethers, or glycosylated derivatives.

It will be interesting to note that the sesquiterpene derivatives with anti-mitotic potentialize the effector thereof.

Such derivatives of the above defined sesquiterpenes with anti-mitotic agents are then in the scope of the invention.

α-bisabolol is a cyclic sesquiterpene having the formula:

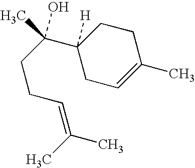

Farnesol is a linear sesquiterpene with the formula:

(CH₃)₂—C—CH—(CH₂)₂—C((CH₃)═CH—(CH₂)₂—C(CH₃)—CH—CH₂OH

Geraniol is also a linear sesquiterpene, having the formula

(CH₃)₂—C—CH—(CH₂)₂—C(CH₃)═CHOH.

According to the invention advantageous pharmaceutical compositions contain in their active principle from 99 to 1% α-bisabolol and respectively 1 to 99% of farnesol and/or geraniol, in particular 90 to 10% α-bisabolol, and 10 to 90% farnesol and/or geraniol, respectively, optionally under the form of their derivatives, such as above defined.

In mixtures composed of x-bisabolol and of farnesol and/or geraniol, α-bisabolol is advantageously present in proportions more or less equal to those of farnesol, or higher.

In particular, tile invention aims at pharmaceutical compositions containing in their active principle 99 to 50% of α-bisabolol and respectively 1 to 50% farnesol, and notably 90 to 50% α-bisabolol and respectively 10 to 50% farnesol, as with geraniol.

In compositions containing farnesol and geraniol mixed with α-bisabolol, geraniol is generally present in lower proportions or at most equal to that of farnesol. These sesquiterpenes are thus, for example, notably in the ratios farnesol/geraniol/α-bisabolol of 35/35/30%, 40/20/40% or 20/40/40%.

The pharmaceutical compositions defined above may also contain one or several other linear or cyclic sesquiterpenes.

For example, they may be sesquiterpenes with a bisabolol ring, such as lanceol, or guaian rings, with the group of guaianoids and derivatives with a patchoulane ring.

These compounds may represent 1 to 5% in the compositions defined above, with the understanding that their proportion is always lower than that of farnesol, of geraniol and of α-bisabolol or their derivatives.

The enantiomeric and distereoisomeric forms of the sesquiterpenes used in the mixtures of the invention are also part of the modes of preparation of the latter.

The levorolary forms are particularly preferred for therapeutic applications, while the other forms are more particularly used as laboratory reagents.

It is known that sesquiterpenes of mixtures according to the invention arc widely used in perfumery and are thus readily available as commercial products. As discussed above, they can also be obtained by synthesis or extraction from plants.

By the use of chemical synthesis, one or several of their —OH groups can be advantageously blocked by a protective group by the use of classical techniques. As a variant, or optionally in combination with the previous disposition, one or several of these —OH groups can be rendered functional to confer special properties to the compounds, in particular to enable their use as pro-drugs. As examples, we can cite esters, in particular acetates, or the esters with methohexate and as above mentioned, ethers, in particular with hydroxylated compounds such as adriamycin, alkyl ethers containing 1 to 4 carbon atoms, or glycosylated derivatives.

Toxicology studies of these compounds have shown their high levels of safety. In this context, it will be noted that α-bisabolol complies with the G.R.A.S. system in the United States. Measures of acute toxicity in male and female mice, as well as in male and female rats (doses administered 600, 800 and 1200 g of a mixture of sesquiterpenes per kg live bodyweight) have shown that the LD₅₀ is much higher than 5 g/kg.

The oral LD₅₀ in male mice is thus 7.5 W kg higher than 9.75 g/kg in male and female rats and 12 g/kg in female mice.

The LD₅₀ measured IP is higher than 1 g/kg (1.25 g/kg in male mice, 1.3 g/kg in female mice, 1.80 g/kg in female rats and 1.90 g/kg in male rats).

Upon autopsy, no organ involvement was observed in the animals.

Long-term nutritional toxicity studies have also been carried out in rats, with control of rood intake and assessment of animal bodyweights.

During periods of 3, 12 and 24 months, rats received a normal diet supplemented with a composition containing sesquiterpenes as defined above at 0.400 g per 100 g of food. Anatomo-pathology examinations revealed no physiological or pathological anomalies. In particular, no spontaneous, tumour development was observed, leading to the conclusion of an absence of long-term nutritional toxicity.

These advantageous properties can be used to fully utilize the synergistic effects, shown in the examples, of the anti-tumour activities of mixtures of sesquiterpenes defined above in nutritional and specific anti-tumour applications in animals And humans.

In these applications, the pharmaceutical applications may contain the active ingredients of other drugs. In particular, their association with water-soluble and fat-soluble vitamins, amino acids such as β-alanine, and anti-tumour drugs will be cited. As examples of anti-mitotic agents that can be used, it will be cited as examples alkylating agents, nitrosoureas, organoplastins, antifolics, anti-purinc and anti-pyrimidine compounds, topoisomerase inhibitors, spindle drugs and hormonal and non-hormonal cytostatic agents. These associations can also contain antibiotics; such as penicillins, cephalosporins, beta-laetamines, aminosides, lincosamides, polymyxins, quinolones, 5-nitiosoamidazoles, sulfamides, teicoplanins, vincomycin (or any other anti-tumour active ingredient).

The pharmaceutical compositions of the invention will also be used advantageously in association with compounds facilitating their assimilation, e.g. sugars such as glucose, proteins such as animal protein hydrolysates, in particular from fish, or lipids such as dietary oils such as rapeseed, sunflower or olive.

It is also advantageous, depending on the localization of the tumour, to combine surgery and or chemotherapy or radiotherapy with the treatment by the compositions according to the invention.

The use of different mixtures results in a decrease in the active chemo- or radiotherapy doses, thereby reducing the more or less toxic iatrogenic side effects.

The pharmaceutical compositions of the invention can be administered in different forms. Thus, they can be administered orally, topically or by injection, sub-cutaneous, intravenous or intramuscular, even rectally.

For oral administration, use is made in particular of ampoules, tablets, pellets, lozenges, capsules, drops, syrups. These compositions advantageously contain from 7 to 25 g of active ingredient per administration unit, preferably 5 to 10 g (equivalent to administration of 0.1 to 0.7 or 0.8 g/kg bodyweight).

For cutaneous administration, in the form of ointments, creams, gels or any other suitable pharmaceutical form, doses are included between 2 and 15 g in one or several applications.

Other forms of administration include solutions injectable intravenously, subcutaneously or intramuscularly, prepared from sterile or sterilizable solutions, and available in different volumes, for example 10, 50, 100, 500 and 1000 ml. They may also be suspensions or emulsions.

These injectable forms contain 10 to 50 g of active, ingredient per administration unit, preferably 10 to 25 g.

Rectal administration uses suppositories, as pharmaceutical form.

The study of the effects of compositions according to the invention in normal rats has shown that mixtures of sesquiterpenes of active ingredients bind and accumulate in tissues with rapid cell renewal.

The compositions of the invention are thus especially suited for treating all types of cancers: digestive, uterine, vaginal, of the pharynx and larynx, melanomas, lymphomas, cancers of the pancreas, lungs, prostate, bladder, bone, etc.

The application of the pharmaceutical compositions of the invention to prevent potential tumour development in the above-mentioned cancers is also within the scope of the invention.

As an indication, the dosage usable in humans to treat mammary tumours or lung cancer, include the following doses: 7 to 25 g/day (0.1 to 0.7 g/kg) of active ingredient containing mixtures of sesquiterpenes, in one or several administrations.

According to another aspect, the invention aims at using mixtures of sesquiterpenes defined above for the preparation of anti-tumour drugs.

It also aims at their use as laboratory reagents to be used as references in studies of the anti-tumour activities of products to test.

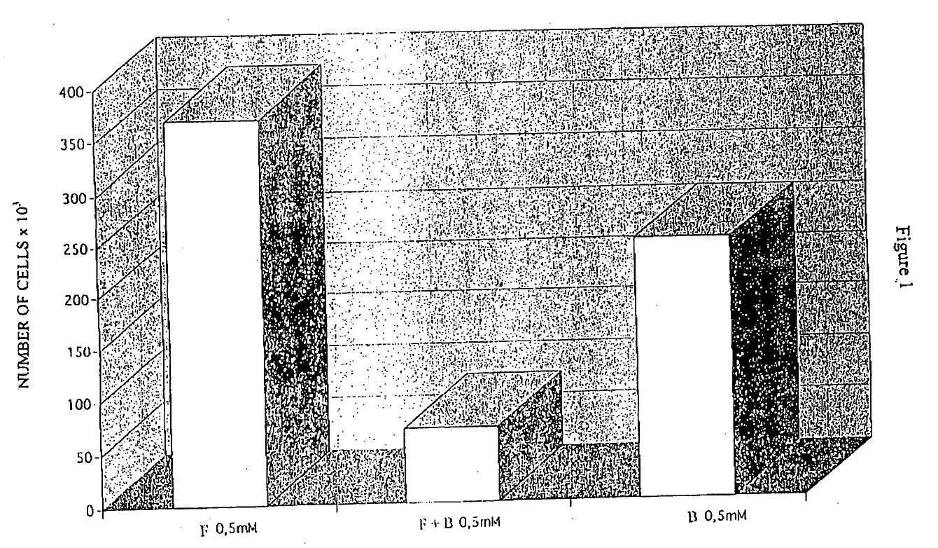

Other characteristics and advantages of the invention will be seen in the examples that follow, with reference to FIGS. 1-6, that show the effect of the following on cultured cells

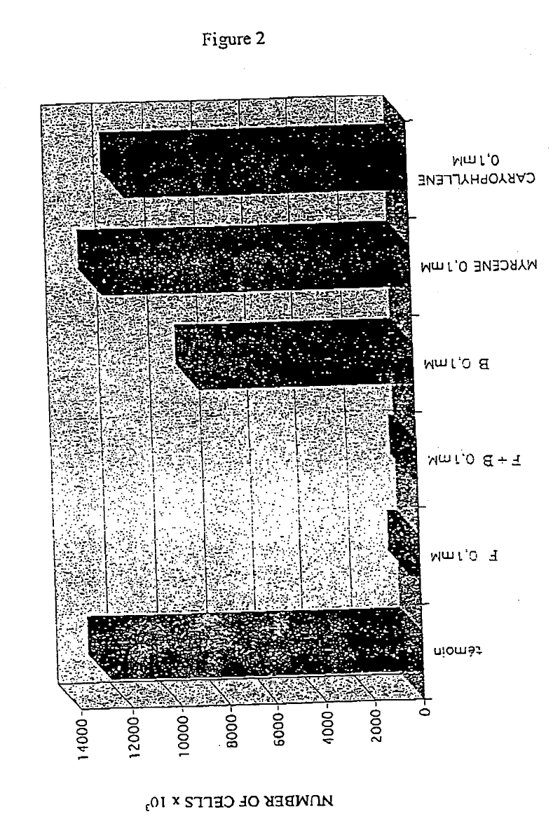

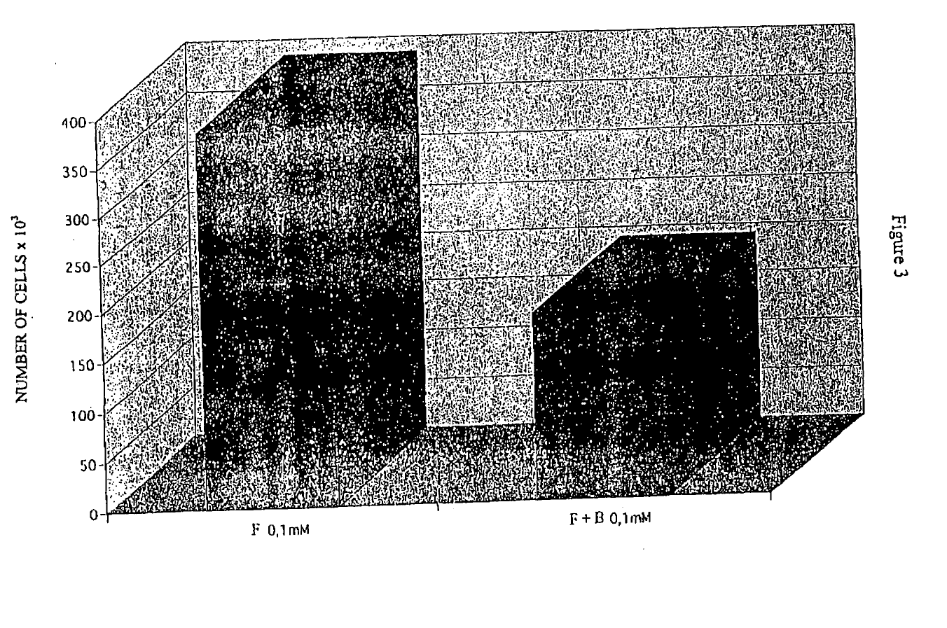

FIGS. 1-3: of a mixture of farnesol and α-bisabolol and of its constituents at 0.5 mM (FIG. 1) and 0.1 mM (FIG. 2: cell count scales 0 to 14,000, FIG. 3: 0 to 400),

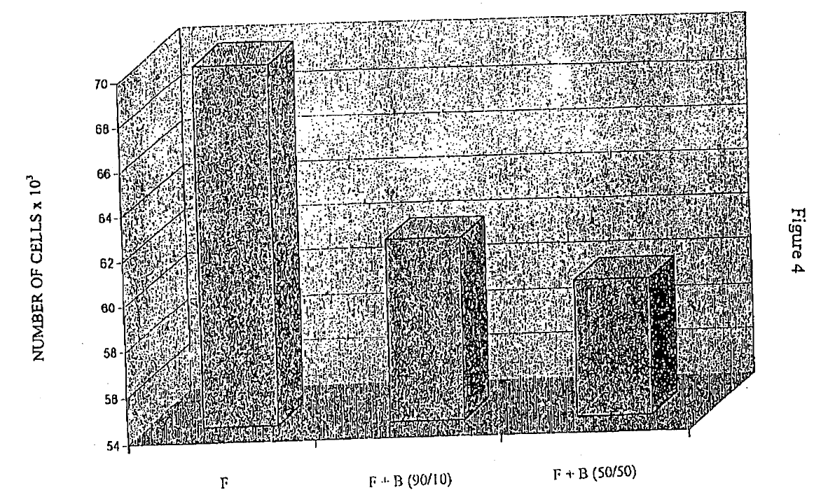

FIG. 4: of a mixture of farnesol and α-bisabolol at different concentrations with respect to farnesol alone,

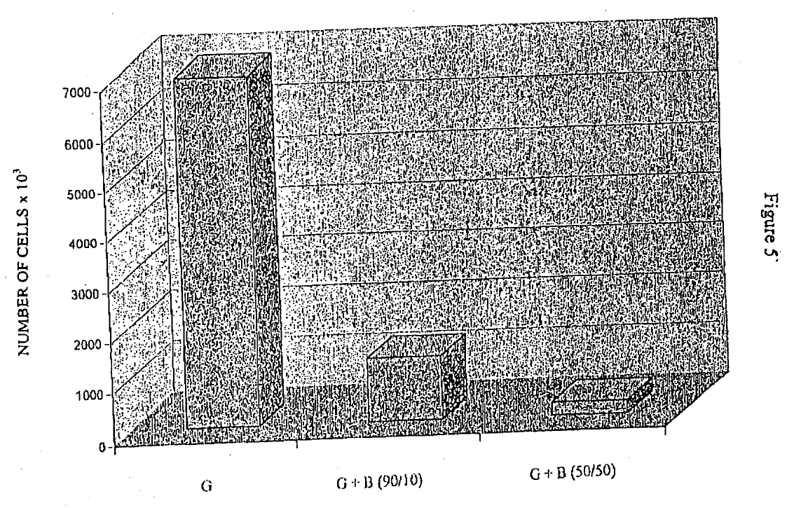

FIG. 5: of a mixture of geraniol and α-bisabolol at different concentrations with respect to geraniol alone and

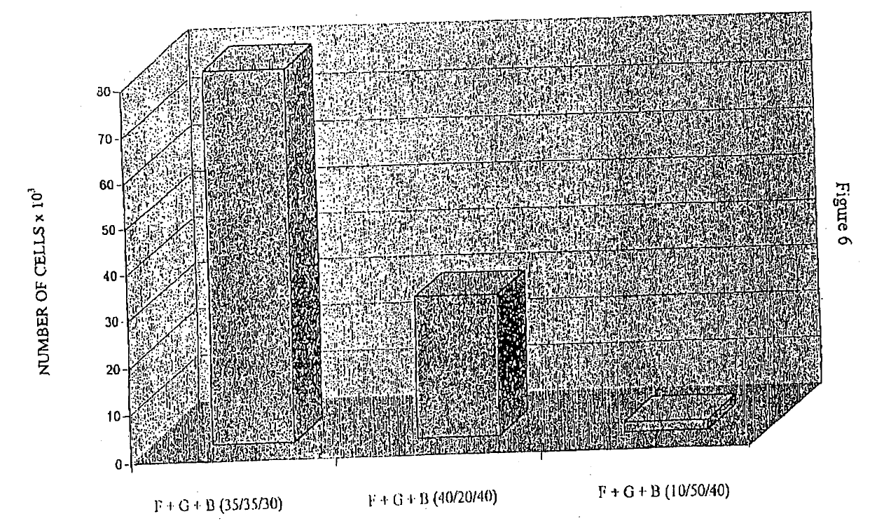

FIG. 6: of a mixture of farnesol/geraniol/α-bisabolol at different concentrations.

### EXAMPLE 1

### Comparative Study of the Activity of Farnesol (F),and a Mixture According to the Invention Farnesol and α-bisabolol (F+B) on Line MCF7

Cells of line MCF7 are rapidly growing mammary adenocarcinoma cells.

The doses tested in the study were 0.1.10⁻³ and 0.5.10⁻³ M.

1/100 dilutions were made in RPMT just before use and mixed to obtain a homogeneous emulsion at the moment of use.

The mean number of cells inoculated was about 10⁶ in each test.

Test one: search for an effect on MCF7 cells at the dose of 0.5 mM.

At the 11^{th} transfer, the following were inoculated

one bottle of MCF7 cells for growth,

one bottle of MCF7 cells for the control,

one bottle with the same number of calls +(F) at 0.5 mM,

one bottle with the same number of cells +(F+B) at 0.5 mM,

one bottle with the same number of cells +(B) at 0.5 mM.

Bottles were placed in the incubator with caps unscrewed and the medium was changed every two days. On day 8, the cells were washed with 5 ml of PBS and freed from the substrate with PBS-EDTA, resvered with 10 ml of RPMI+10% FCS and counted with a Malassez counting chamber.

Test two: search of an effect on MCF7 cells at a dose of 0.1 mM.

Same procedure as above is used but cells were used at the 12^{th} transfer, using 0.1 mM doses of (F), (F+B) and (B).

Results

Test one: Test at the dose of 0.5 mM (the results are shown in FIG. 1),

bottles of cells in culture presented an adhering homogeneous layer of cells with several dead cells; the medium was orange,

the bottle containing 0.5 mM F contained few adhering cells and a large number of floating (dead) cells); the medium was moderate to strong violet,

the bottle containing the mixture F+B at 0.5 mM contained large numbers of agglomerated, floating (dead) cells; the medium was moderate to strong violet; there were only rare adhering cells,

the bottle containing B at 0.5 mM contained few adhering cells and a large numbers of floating (dead) cells; the medium was moderate to strong violet. This appearance was comparable to that of the culture with F alone.

The following cell counts were obtained:

| | |
|---|---|
| transfer control: | 12,750 · 10³ |
| test control: | 13,500 · 10³ |
| F alone at 0.5 mM: | 370 · 10³ |
| mixture F + B at 0.5 mM (50/50): | 70 · 10³ |
| B at 0.5 mM: | 250 · 10³ |

These results arc shown in FIG. 1.

Test two: Test at the dose of 0.1 mM

the bottle of cells in culture (control) presented an adhering homogeneous layer of cells with several dead cells; the medium was orange,

the bottle containing B presented an adhering homogeneous layer of cells with several dead cells; the medium was orange. This appearance was similar to that of the control bottle,

the bottle containing F alone at 0.1 mM contained few adhering cells and a large numbers of floating (dead) cells; the medium was moderate to strong violet,

the bottle containing the mixture F+B at 0.1 mM contained few adhering cells and a large numbers of floating (dead) cells; the medium was moderate to strong violet.

The following cell counts were obtained:

| | |
|---|---|
| test control: | 12,780 · 10³ |
| F alone at 0.1 mM: | 385 · 10³ |
| mixture F + B at 0.1 mM (50/50): | 190 · 10³ |
| B at 0.5 mM: | 8930 · 10³ |

The results show an alteration of MFC7 cell adherence in culture with F alone, F+B, and B alone at the dose of 0.5 mM. The same was found in the test run at 0.1 mM for F alone and the mixture. The change was slight with B. Counting showed that cell numbers were lower than those obtained with the controls, but the macroscopic appearance of the culture bottle was comparable to that of the test bottle.

In both tests, the mixture F+B furnished a greater alteration than that obtained with F alone, regardless of the dose.

In these tests, the activity of the mixture F+B against MCF7 mammary adenocarcinoma cells in culture was higher than that of F alone or B alone at the same dose.

These results are shown in the graphs of FIGS. 2 and 3 at the cell count scales of 0 to 14,000 and 0 to 400, respectively.

### EXAMPLE 2

### Comparative Study of Activity of Farnesol (F) or Geraniol (G), and Mixtures According to the Invention of Farnesol and α-bisabolol (F+B) or Geraniol α-Bisabolol (G+B) on RIN 5 mF Cells

The conditions described in example 1 were used. The following results were obtained:

a—Effect of F and of F+B:

| Product (0.1 mM) | cell numbers (× 10³) |
|---|---|
| F at 0.1 mM | 70 · 10³ |
| F + B (90/10) | 62 · 10³ |
| F + B (50/50) | 60 · 10³ |
| control | 12,000 · 10³ |

These results, also shown in FIG. 4, show that the mixture according to the invention, at different doses, reduces the number of cells by at least 10% in the conditions of the test in comparison to farnesol alone.

b—Effect of G and of G+B:

| Product (0.1 mM) | cell number (× 10³) |
|---|---|
| G at 0.1 mM | 6900 · 10³ |
| G + B (90/10) | 1240 · 10³ |
| G + B (50/50) | 245 · 10³ |
| control | 11,600 · 10³ |

Again we seen the advantageous effect of the mixtures according to the invention, in particular at the proportion 50/50 (also see FIG. 5).

It is of interest to note that in studies of cultured cells, where tumoural pancreatic cells were mixed with non-tumoural pancreatic cells, the different compositions of farnesol, geraniol and α-bisabolol selectively destroyed only the tumoural pancreatic cells.

c—Effect of F+G+B:

| Product (0.1 mM) | cell numbers (× 10³) |
|---|---|
| F + G + B (35/35/30) | 80 · 10³ |
| F + G + B (40/20/40) | 30 · 10³ |
| F + G + B (20/40/40) | 2 · 10³ |
| control | 10,850 · 10³ |

These results demonstrate the anti-mitotic effect of mixtures of the invention, particularly advantageous with ternary mixture rich in α-bisabolol.

### EXAMPLE 3

### Application of Mixtures According to the Invention to Patients with Cancers Chop Treatment: (1 Week)

The following products were administered to the patient:

Oncovin®: 0.6 to 1.4 mg/m²

Adriamycin®: 50 to 300 mg/d

Endoxan®: 100 to 500 mg/m²

Prednisone®: usual treatment

Coupled treatment (1 week):

Deticine®: 50 to 250 g/m²

Doxyrubicin®: 10 to 75 mg/m²

Short cycle (3-4 days)

Zophrene®: 1 of ½ tablet, 4 to 8 mg/d

combined with

Holoxan®: 0.5 and 3 g/m²

Cisplatin: 10 to 120 mg/m²

Pionalide®: 100 and 400 mg/d

The beneficial effects of the mixture according to the invention can be observed in the context of clinical trials on cancer patient after their informed consent. These trials confirmed the absence of toxicity of the mixtures after 6 months of treatment and revealed the disappearance of bone metastases in 4 to 6 months (MRI and bone scans).

As examples of cure within 6 months in 6 cancer patients: one generalized cancer treated with chemotherapy for one year; one cancer of the vocal cords; one breast cancer with bone metastases (after treatment analyses showed the absence of nodules and the disappearance of the metastases); one lymphoma; one bladder tumour with metastases of the sacrum.

1/ Pharmaceutical compositions comprising possibly in association with an inert pharmaceutical vehicle, a therapeutically effective amount of a mixture of α-bisabolol and farnesol and/or geraniol, and/or the derivatives thereof, particularly of functional derivatives such as esters, for example with methohexate, their ethers, particularly with hydroxylated compounds such as adriamycin, C1-C4 alkyl ethers, or glycosylated derivatives.

2/ Pharmaceutical compositions according to claim 1, comprising in their active principle from 99 to 1% α-bisabolol and respectively 1 to 99% farnesol and/or geraniol, and notably 90 to 10% α-bisabolol and respectively, 10 to 90% farnesol and/or geraniol.

3/ Pharmaceutical compositions according to claim 2, comprising in their active principle a mixture of α-bisabolol and farnesol, α-bisabolol being present in a proportion at least equal to that of farnesol or higher.

4/ Pharmaceutical compositions according to claim 3, comprising in their active principle from 99 to 50% α-bisabolol and respectively 1 to 50% farnesol and notably 90 to 50% α-bisabolol and respectively 10 to 50% farnesol and/or geraniol.

5/ Pharmaceutical compositions according to claim 2, wherein the proportion of geraniol is lower or at most equal to that of farnesol in compositions containing these two sesquiterpenes.

6/ Pharmaceutical compositions according to claim 5, comprising farnesol, geraniol and α-bisabolol in the ratios of 35/35/30, 40/20/40 or 20/40/40.

7/ Pharmaceutical compositions according to any one of claims 1 to 6, further comprising one or several linear or cyclic sesquiterpenes.

8/ Pharmaceutical compositions according to any one of claims 1 to 7, wherein said sesquiterpenes are in the form of derivatives with anti-mitotic agents.

9/ Pharmaceutical compositions according to any one of claims 1 to 8, wherein said sesquiterpenes are in the form of their enantiomers and/or diastereoisomers, in particular in the levorotary form.

10/ Pharmaceutical compositions according to any one of claims 1 to 9, which can be administered orally, topically, by injection or rectally.

11/ Pharmaceutical compositions according to claim 10, intended for administration via the oral route and supplied in the form of pellets, tablets, capsules, lozenges, drops, syrups and that they contain from 7 to 25 g of active ingredient per administration unit, preferably 5 to 10 g.

12/ Pharmaceutical compositions according to claim 10, intended for administration via the cutaneous route and are supplied in the form of an ointment, cream or gel, the doses being included between 2 and 15 g, and in one or several applications.

13/ Pharmaceutical compositions according to claim 10, characterized in that they are injectable solutions, via the intravenous, subcutaneous, or intramuscular route, the solutions advantageously containing 7 to 50 mg of active ingredient per administration unit, preferably 10 to 25 g.

14/ Pharmaceutical compositions according to claim 10, intended for administration via the rectal route and are supplied in the form of suppositories.

15/ Pharmaceutical compositions according to any one of claims 1 to 14, used as anti-tumour drugs to treat all types of cancers of epithelial and connective tissue.

16/ Utilization of mixtures of sesquiterpenes, defined in any one of claims 1 to 9, for the preparation of drugs to treat all types of cancers of epithelial and connective tissue.
